(19) United States
(12) Patent Application Publication
(10) Pub. No.: US 2021/0179516 A1
(43) Pub. Date: Jun. 17, 2021

(54) METHODS AND SYSTEMS USING A REACTOR EFFLUENT EXPANDER FOR OLEFIN PRODUCTION

(71) Applicant: SABIC Global Technologies B.V., Bergen op Zoom (NL)

(72) Inventor: Joris VAN WILLIGENBURG, Maastricht (NL)

(21) Appl. No.: 16/761,685

(22) PCT Filed: Nov. 13, 2018
(86) PCT No.: PCT/IB2018/058931

§ 371 Date: May 5, 2020

Related U.S. Application Data
(60) Provisional application No. 62/590,729, filed on Nov. 27, 2017.

(51) Int. Cl. C07C 5/327; C07C 4/04; B01J 19/00

(52) U.S. Cl. CPC C07C 5/327; C07C 4/04; B01J 2219/00162; B01J 2219/00092; B01J 2219/00081; B01J 19/0013

(57) ABSTRACT

Methods and systems for improving energy conversion from the heat available in a hydrocarbon feedstream during the production of olefins. In a particular non-limiting embodiment, a method can include increasing the temperature of a hydrocarbon feedstream and a hydrogen gas feedstream via a first heat exchanger; combining and feeding the feedstreams into a reactor to produce a reactor effluent including one or more olefins; expanding the reactor effluent in a reactor effluent expander to decrease the pressure and/or temperature of the reactor effluent; transferring the reactor effluent to the heat exchanger to increase the temperature of the feedstreams and/or decrease the temperature of the reactor effluent; and compressing the reactor effluent in a compressor, where the expansion of the reactor effluent drives the compressor.

### CROSS-REFERENCE TO RELATED APPLICATIONS

This application claims the benefit of priority of U.S. Provisional Patent Application No. 62/590,729, filed Nov. 27, 2017, which is hereby incorporated by reference in its entirety.

### FIELD

The presently disclosed subject matter relates to techniques for improving energy conversion during the production of the olefins by the use of a reactor effluent expander.

### BACKGROUND

Many petrochemical processes are endothermic processes and energy can be required to drive such processes. In particular, the input of heat to perform such petrochemical processes can be greater than the amount of heat that is available through heat recovery, which can result in the consumption of high quality energy and be cost-intensive. For example, cracking of hydrocarbons to produce olefins is an endothermic process and requires energy, which can be in the form of high temperature heat to drive the reaction within the reactor, and mechanical work, which can be used to drive the compressors used for product separation.

Methods and systems for reducing the amount of energy consumed during petrochemical processes are known in the art. For example, G.B. Patent No. 1,516,362 discloses a process for producing hydrogen-rich gas in a reformer furnace that includes the generation of electrical power and compressed air by passing furnace flue gas and hot combustion gases through the turbine side of an air compressor in common drive with a generator. WO 2007/143776 discloses a process for generating synthesis gas that includes using the outlet flue gas from a reactor to drive an expansion turbine. Indian Application No. 3225/CHE/2008 discloses a method of recovering energy from a fluid catalytic cracking (FCC) unit having a reactor and a regenerator, where a turbo-expander train can be used to combust and expand the generated syngas to drive a first compressor. The turbo-expander train can include a first expander coupled to a first compressor via a shaft, and the energy required to rotate the shaft can be extracted from the heated gas produced in the combustion zone by the first expander.

U.S. Pat. No. 5,114,682 discloses a process for the recovery of heat energy during a FCC catalyst regeneration process that includes expanding the effluent gases from the catalyst regeneration process to obtain work energy that can be used to operate an expansion turbine-compressor. Krishnasing et al., American Institute of Chemical Engineering Conference (2010), discloses how turbo-expanders convert energy to enhance and optimize cryogenic recovery of ethylene and hydrogen in an ethylene plant.

There remains a need in the art for techniques that can reduce the amount of net energy that is consumed during the formation of olefin containing hydrocarbon streams.

### SUMMARY OF THE DISCLOSED SUBJECT MATTER

The presently disclosed subject matter provides methods and systems for olefin production from a hydrocarbon feedstream, e.g., by hydropyrolysis, including improving energy conversion from the heat available in the hydrocarbon feedstream. In certain embodiments, exemplary methods include the use of a reactor effluent expander in the absence of a reactants expander.

An example method for producing olefins using a hydrocarbon feedstream and a gas feedstream containing hydrogen can include increasing the temperature of the hydrocarbon feedstream and the hydrogen gas feedstream by a first heat exchanger. The method can further include combining the feedstreams and feeding the combined feedstream into a reactor to produce a reactor effluent that includes one or more olefins. In certain embodiments, the method can include expanding the reactor effluent in a reactor effluent expander to decrease the pressure and/or temperature of the reactor effluent. The method can include transferring the expanded reactor effluent to the first heat exchanger to increase the temperature of the feedstream and/or decrease the temperature of the reactor effluent, followed by the compression of the reactor effluent in a compressor. The expansion of the reactor effluent can drive the compressor.

In certain embodiments, the method can further include expanding the combined feedstream in a reactants expander to decrease the pressure and/or decrease the temperature of the feedstream prior to feeding the feedstream into the reactor. In certain embodiments where the combined feedstream is expanded, the expansion of the feedstream can be used to drive the compressor. In certain embodiments, the method can further include separating the olefins from the reactor effluent to produce an olefin-containing stream.

In certain embodiments, the hydrocarbon feedstream can include ethane and/or the reactor effluent can include ethylene. In a non-limiting embodiment, the hydrocarbon feedstream can include propane and/or the reactor effluent can include propylene. In certain embodiments, the gas feedstream can further include a gas such as steam, nitrogen or a combination thereof. In certain embodiments, the hydrocarbon feedstream can include butane and/or the reactor effluent can include 1-butene, isobutylene, hydrogen or a combination thereof. In a non-limiting embodiment, the hydrocarbon feedstream can be a C4 hydrocarbon stream and/or the reactor effluent can include 1,3-butadiene. In certain embodiments, the gas feedstream and/or the hydrocarbon feedstream can have a pressure of about 10 to 50 bar absolute and/or a temperature of about 10° C. to about 100° C. prior to increasing the temperature of the hydrocarbon feedstream and the hydrogen gas feedstream via the first heat exchanger.

The presently disclosed subject matter further provides methods for producing ethylene using a feedstream including ethane and hydrogen. In certain embodiments, the method includes increasing the temperature of the feedstream via a first heat exchanger to a temperature equal to or greater than about 500° C. The method can further include increasing the temperature of the feedstream to about 500° C. to about 700° C. via a second heat exchanger. The method can further include feeding the feedstream into a reactor and cracking the feedstream at a temperature from about 700° C. to about 880° C. to produce a reactor effluent that includes ethylene at a temperature from about 700° C. to about 880° C. and/or at a pressure from about 1 to about 5 bar absolute. In certain embodiments, the method can include expanding the reactor effluent in a reactor effluent expander to decrease the pressure of the reactor effluent to about 0.2 to about 1.2 bar absolute and/or to decrease the temperature of the reactor effluent to a temperature from about 600° C. to about 700° C. In a non-limiting embodiment, the method can include decreasing the temperature of the reactor effluent in the first heat exchanger and compressing the reactor effluent in a compressor to a pressure from about 0.3 bar to about 35 bar. In certain embodiments, the expansion of the reactor effluent drives the compressor.

In certain embodiments, the method can further include expanding the combined feedstream in a reactants expander to decrease the pressure and/or decrease the temperature of the feedstream prior to increasing the temperature of the feedstream in a second heat exchanger. In certain embodiments, the method can include separating ethylene from the compressed reactor effluent to produce an ethylene product stream. In certain embodiments, the temperature of the reactor effluent is about 20° C. to 30° C. prior to the compression of the reactor effluent.

The presently disclosed subject matter further provides a system for producing olefins using a hydrocarbon feedstream and a gas feedstream including hydrogen. The system can include a first heat exchanger for increasing the temperature of the hydrocarbon feedstream and the hydrogen gas feedstream. The system can include a second heat exchanger, coupled to the first heat exchanger, for increasing the temperature of the feedstreams. The system can further include a reactor, coupled to the second heat exchanger, for reacting the feedstreams to produce a reactor effluent from the feedstreams, and a reactor effluent expander, coupled to the reactor and the first heat exchanger, for decreasing the temperature and/or pressure of the reactor effluent. The system can include a third heat exchanger, coupled to the first heat exchanger, for decreasing the temperature of the reactor effluent and a compressor, coupled to the third heat exchanger, for compressing the reactor effluent. In certain embodiments, the reactor effluent expander and the compressor within the system can be coupled and the expansion of the reactor effluent can drive the compressor. In certain embodiments, the system can further include a reactants expander, coupled to the first heat exchanger and the second heat exchanger, for decreasing the pressure and/or temperature of the feedstreams before entering the second heat exchanger. In certain embodiments, the reactants expander is coupled to the compressor and expansion of the feedstreams drives the compressor. In certain embodiments, the reactants expander, the reactor effluent expander and the compressor are coupled and the expansion of the feedstreams and the reactor effluent drives the compressor. The following includes definitions of various terms and phrases used throughout this specification.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The term “bulk metal catalyst” as that term is used in the specification and/or claims, means that the catalyst includes one metal, and does not require a carrier or a support.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The processes of the present invention can “comprise,” “consist essentially of” or “consist of” particular ingredients, components, compositions, etc. disclosed throughout the specification.

In the context of the present invention, twenty embodiments are now described. Embodiment 1 is a method for producing olefins using a hydrocarbon feedstream and a gas feedstream including hydrogen, including the steps of (a) increasing the temperature of the hydrocarbon feedstream and the gas feedstream via a heat exchanger; (b) combining the hydrocarbon feedstream and the gas feedstream and reacting the feedstreams to produce a reactor effluent comprising one or more olefins; (c) expanding the reactor effluent in a reactor effluent expander to decrease one or more of a reactor effluent pressure or reactor effluent temperature; (d) transferring the reactor effluent to the heat exchanger to increase the temperature of the feedstreams and/or decrease the temperature of the reactor effluent; and (e) compressing the reactor effluent by utilizing reactor effluent expansion. Embodiment 2 is the method of embodiment 1, wherein the hydrocarbon feedstream comprises ethane and/or the reactor effluent contains ethylene. Embodiment 3 is the method of embodiment 1, wherein the hydrocarbon feedstream contains propane and/or the reactor effluent contains propylene. Embodiment 4 is the method of any of embodiments 1 to 3, further including the step of expanding the combined feedstream in a reactants expander to decrease the pressure and/or the temperature of the combined feedstream prior to feeding the combined feedstream into the reactor. Embodiment 5 is the method of any of embodiments 1 to 4, further including the step of separating the olefins from the reactor effluent to generate an olefin-containing stream. Embodiment 6 is the method of any of embodiments 1 to 5, wherein the gas feedstream further includes a gas selected from the group consisting of steam, nitrogen, methane, hydrogen, carbon dioxide and a combination thereof. Embodiment 7 is the method of any of embodiments 1 to 6, wherein the gas feedstream and/or the hydrocarbon feedstream has a pressure of about 10 to 50 bar absolute and/or a temperature of about 10° C. to about 100° C. prior to increasing the temperature of the hydrocarbon feedstream and the hydrogen gas feedstream via a first heat exchanger.

Embodiment 8 is a method for producing ethylene using a feedstream including ethane and hydrogen, the method including the steps of (a) increasing the temperature of the feedstream via a heat exchanger to a temperature equal to or greater than about 500° C.; (b) feeding the feedstream into a reactor and increasing the temperature of the hydrocarbon feedstream to a temperature from about 500° C. to about 700° C.; (c) cracking the feedstream at a temperature from about 650° C. to about 880° C. to produce a reactor effluent comprising ethylene at a temperature from about 780° C. to about 880° C. and/or a pressure from about 2 to about 5 bar absolute; (d) expanding the reactor effluent in an reactor effluent expander to decrease one or more of a pressure of the reactor effluent to about 0.2 to about 1.2 bar absolute or reactor effluent temperature to about 600° C. to about 700° C.; (e) transferring the reactor effluent to the heat exchanger to increase the temperature of the feedstream and/or decrease the temperature of the reactor effluent; and (f) compressing the reactor effluent by utilizing reactor effluent expansion. Embodiment 9 is the method of embodiment 8 further including the step of expanding the feedstream in a reactants expander to decrease the pressure and/or the temperature of the feedstream prior to feeding the feedstream into the reactor. Embodiment 10 is the method of any of embodiments 8 or 9, further including the step of separating ethylene from the compressed reactor effluent to produce an ethylene product stream. Embodiment 11 is the method of any of embodiments 8 to 10, wherein the temperature of the reactor effluent is about 20° C. to about 50° C. prior to compressing the reactor effluent. Embodiment 12 is a method for producing ethylene using a feedstream including ethane and hydrogen, including the step of (a) increasing the temperature of the feedstream via a heat exchanger; (b) feeding the feedstream into a reactor and increasing the temperature of the feedstream; (c) cracking the feedstream at a temperature from about 650° C. to about 880° C. to produce a reactor effluent containing ethylene; (d) expanding the reactor effluent in an reactor effluent expander to decrease one or more of a reactor effluent pressure or reactor effluent temperature; (e) transferring the reactor effluent to the heat exchanger to increase the temperature of the feedstream and/or decrease the temperature of the reactor effluent; (f) separating one or more condensed components from the reactor effluent; and (g) compressing the reactor effluent by utilizing reactor effluent expansion. Embodiment 13 is the method of embodiment 12 further including the step of separating ethylene from the compressed reactor effluent to produce an ethylene product stream. Embodiment 14 is the method of any of embodiments 12 or 13 further including the step of decreasing the pressure and/or temperature of the feedstream in a reactants expander before feeding the feedstream into a reactor. Embodiment 15 is a system for producing olefins using a hydrocarbon feedstream and a gas feedstream including hydrogen, including (a) a first heat exchanger for increasing the temperature of the hydrocarbon feedstream and the hydrogen gas feedstream; (b) a second heat exchanger, coupled to the first heat exchanger, for increasing the temperature of the feedstreams; (c) a reactor, coupled to the second heat exchanger, for producing a reactor effluent from the feedstreams; (d) a reactor effluent expander, coupled to the reactor and the first heat exchanger, for decreasing the temperature and/or pressure of the reactor effluent; (e) a third heat exchanger, coupled to the first heat exchanger, for decreasing the temperature of the reactor effluent; and (f) a compressor, coupled to the third heat exchanger and the reactor effluent expander, for compressing the reactor effluent in a compressor, wherein the expansion of the reactor effluent drives the compressor. Embodiment 16 is the system of embodiment 15, further including a reactants expander, coupled to the first heat exchanger and the second heat exchanger, for decreasing the pressure and/or temperature of the feedstreams before entering the second heat exchanger. Embodiment 17 is the system of any of embodiments 15 or 16, wherein the system does not include a reactants expander. Embodiment 18 is a system for producing olefins using a hydrocarbon feedstream and a gas feedstream including hydrogen, including (a) a first heat exchanger for increasing the temperature of the hydrocarbon feedstream and the hydrogen gas feedstream; (b) a reactants expander, coupled to the first heat exchanger, for decreasing the pressure and/or temperature of the feedstreams; (c) a second heat exchanger, coupled to the reactants expander, for increasing the temperature of the feedstreams via a second heat exchanger; (d) reactor, coupled to the second heat exchanger, for producing a reactor effluent from the feedstream; (e) a reactor effluent expander, coupled to the reactor and the first heat exchanger, for decreasing the temperature and/or pressure of the reactor effluent; (f) a third heat exchanger, coupled to the first heat exchanger and the reactor effluent expander, for decreasing the temperature of the reactor effluent; and (g) a compressor, coupled to the third heat exchanger, the reactor effluent expander and the reactants expander, for compressing the reactor effluent in a compressor, wherein the expansion of the feedstreams and/or the reactor effluent drives the compressor. Embodiment 19 is the system of embodiment 18, wherein the reactants expander is coupled to the compressor and expansion of the feedstreams drives the compressor. Embodiment 20 is the system of any of embodiments 18 or 19, wherein the reactants expander, the reactor effluent expander and the compressor are coupled and the expansion of the feedstreams and the reactor effluent drives the compressor.

### BRIEF DESCRIPTION OF THE DRAWINGS

FIG. 1 is a schematic diagram depicting an exemplary system in accordance with one non-limiting embodiment of the disclosed subject matter.

FIG. 2 is a schematic diagram depicting an exemplary method in accordance with one non-limiting embodiment of the disclosed subject matter.

### DETAILED DESCRIPTION

The presently disclosed subject matter provides methods and systems for olefin production from a hydrocarbon feedstream, e.g., by hydropyrolysis, and for improving energy conversion from the heat available in the hydrocarbon feedstream. In certain embodiments, the methods and systems of the present disclosure use a reactor effluent expander for recovering energy from the reactor effluent as mechanical work, e.g., which can be used to drive other components within the system, and to minimize the energy expended during the production of olefin-containing hydrocarbon streams. For example, and not by way of limitation, the disclosed methods can be used to generate olefins such as, but not limited to, ethylene.

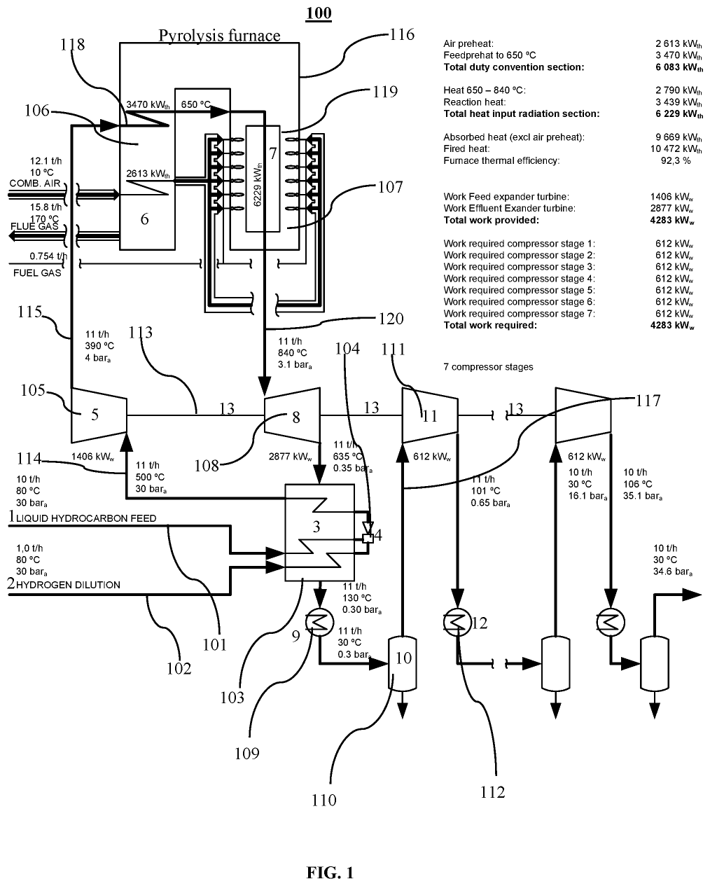

For the purpose of illustration and not limitation, FIG. 1 is a schematic representation of a system according to a non-limiting embodiment of the disclosed subject matter. In certain embodiments and as shown in FIG. 1, the system 100 can include a process furnace 116. The furnace for use in the present subject matter can be any furnace known in the art. For example, and not by way of limitation, the furnace 116 is a pyrolysis furnace and can include a radiant section 107 and a convection section 106.

In certain embodiments, the system 100 can include one or more feed lines, e.g., 101 and 102, coupled to a heat exchanger 103. For example, and not by way of limitation, feed line 101 can be used to feed a hydrocarbon feedstream to the heat exchanger 103. Alternatively or additionally, feed line 102 can be used to transfer a second feedstream and/or diluent, e.g., a hydrogen stream, to the heat exchanger 103. The heat exchangers of the present disclosure can be of various designs known in the art. In certain embodiments, the heat exchangers can be double pipe exchangers. In certain embodiments, the heat exchangers can include a bundle of tubes housed in a shell, such that streams to be warmed or cooled within the heat exchanger flow through the shell and/or bundle of tubes. In certain embodiments, the heat exchangers can include corrosion-resistant materials. In certain embodiments, the heat exchangers can include an alloy, e.g., steel or carbon steel. In certain embodiments, the heat exchangers can include brazed aluminum.

“Coupled” as used herein refers to the connection of a system component to another system component by any means known in the art. The type of coupling used to connect two or more system components can depend on the scale and operability of the system. For example, and not by way of limitation, coupling of two or more components of a system can include one or more joints, valves, transfer lines or sealing elements. Non-limiting examples of joints include threaded joints, soldered joints, welded joints, compression joints and mechanical joints. Non-limiting examples of fittings include coupling fittings, reducing coupling fittings, union fittings, tee fittings, cross fittings and flange fittings. Non-limiting examples of valves include gate valves, globe valves, ball valves, butterfly valves and check valves.

In certain embodiments, the system 100 can further include a chamber 104, e.g., mixing chamber, coupled to the heat exchanger 103. The chamber 104 can be used for combining the multiple feedstreams fed into the heat exchanger 103, e.g., by feed lines 101 and 102, into a single feedstream after the initial heat exchange.

The system 100 can further include a second heat exchanger 118. In certain embodiments, the second heat exchanger 118 can be coupled to the first heat exchanger 103. The second heat exchanger 118 can be located within the convection section 106 of the furnace 116. In certain embodiments, the feedstream can be transferred from the first heat exchanger 103 to the second heat exchanger 118 to increase the temperature of the feedstream by exchanging heat with one or more gases within the furnace. For example, and not by way of limitation, the feedstream can exchange heat with one or more flue gases originating from the radiant section 107 of the furnace 116.

In certain embodiments, the system 100 can further include a reactor 119 coupled to the second heat exchanger 118. In certain embodiments, the reactor 119 can be any reactor that can be operated at low pressures, e.g., at pressures below about 45 bar gauge (barg), and/or at high temperatures, e.g., at temperatures higher than about 500° C. In certain embodiments, the reactor 119 can be a reactor that is used to produce olefins, e.g., by cracking hydrocarbon containing feedstreams. In certain embodiments, the reactor 119 can be a fluidized catalytic cracker. In certain embodiments, the reactor 119 can be a reactor used to produce propylene, butylene, 1,3-butadiene and/or ethylene. In certain embodiments, the reactor 119 can be a reactor used for steam reforming for the production of hydrogen, e.g., methane steam reforming to produce hydrogen-containing syngas. In certain embodiments, the reactor 119 can be a reactor used for hydrocracking. For example, and not by way of limitation, the reactor 119 can be a reactor used for mild hydrocracking of pyrolysis gas to generate aromatics such as, but not limited to, benzene, toluene, xylene and/or ethylbenzene.

In certain embodiments, the reactor 119 can be coupled to an expansion element 108 for expanding the reactor effluent (referred to herein as a reactor effluent expander). In certain embodiments, the reactor 119 can be coupled to the reactor effluent expander 108 via a transfer line 120, e.g., to transfer the effluent from the reactor to the reactor effluent expander 108. In certain embodiments, the reactor effluent can be expanded within the reactor effluent expander 108 to reduce its pressure and/or decrease its temperature. The reactor effluent expander can be used to extract work from the heat of the reactor effluent to drive other components within the system.

In certain embodiments, the reactor effluent expander 108 can be coupled to a heat exchanger to further reduce the temperature of the reactor effluent. For example, and not by way of limitation, the reactor effluent expander 108 can be coupled to the first heat exchanger 103 to exchange heat between the reactor effluent and the feedstreams within the first heat exchanger 103. In certain embodiments, the system 100 can include a third heat exchanger, e.g., for further reducing the temperature of the reactor effluent. In certain embodiments, the first heat exchanger 103 can be coupled to a third heat exchanger 109.

In certain embodiments where condensation of components within the reactor effluent occurs following cooling, e.g., after heat exchange in the first and/or third heat exchangers, the system 100 can further include one or more separation units 110 for separating the condensed components from the reactor effluent. The separation unit 110 can be coupled to the third heat exchanger 109.

In certain embodiments, the system 100 can further include one or more compressors 111. For example, and not by way of limitation, the system 100 can include a booster compressor and/or a gas compressor. In certain embodiments, the system 100 can include one or more booster compressors and one or more gas compressors. In certain embodiments, the use of a booster compressor can reduce the duty of a gas compressor that is downstream of the booster compressor. The compressor 111 can be coupled to the separation unit 110 via a transfer line 117 and/or coupled to the third heat exchanger 109 to increase the pressure of the reactor effluent and/or further reduce the temperature of the reactor effluent, e.g., to prepare the reactor effluent for downstream separation processes. The compressor 111 can include one or more stages and/or one or more cooling units 112 (see FIG. 1).

In certain embodiments, the system 100 can further include a second expansion element 105, e.g., an expander or an expansion turbine, for expanding the feedstream (referred to herein in as a reactants expander). For example, and not by way of limitation, the heat exchanger 103 can be coupled to the reactants expander 105 via a transfer line 114, e.g., to transfer the feedstream, e.g., the combined feedstream as disclosed above, from the heat exchanger 103 to the reactants expander 105. In certain embodiments, the reactants expander 105 can be used to reduce the pressure and/or temperature of the feedstream and for extracting work from the heat of the feedstream. In certain embodiments, the reactants expander 105 can be coupled to the second heat exchanger 118, e.g., via transfer line 115. In certain embodiments, the expanded feedstream can be transferred to the second heat exchanger 118 from the expander 105 via transfer line 115. In certain embodiments, the second heat exchanger 118 can be coupled to both a reactants expander and the first heat exchanger 103, e.g., to allow the partial bypass of the reactant expander to control the heat balance around the furnace and/or the amount of generated work produced by the expanders. In certain embodiments, the system 100 of the present disclosure does not include a reactants expander.

In certain embodiments where the system 100 includes a reactants expander 105 and a reactor effluent expander 108, both expanders can be used to extract heat from the reactants and the reactor effluent, respectively, to drive other components within the system. In certain embodiments, the reactants expander 105, if present within the system, the reactor effluent expander 108 and/or the one or more stages of the compressor 111 can be mounted on the same axis 113 to allow for the transfer of mechanical work between these components. For example, and not by way of limitation, the compressor 111 can be coupled to the reactor effluent expander 108 and/or the reactants expander 105.

The system of the present disclosure can further include additional components and accessories including, but not limited to, one or more additional feed lines, gas exhaust lines, cyclones, product discharge lines, reaction zones, heating elements and one or more measurement accessories. The one or more measurement accessories can be any suitable measurement accessory known to one of ordinary skill in the art including, but not limited to, pH meters, pressure indicators, pressure transmitters, thermowells, temperature-indicating controllers, gas detectors, analyzers and viscometers. The components and accessories can be placed at various locations within the system.

In accordance with the embodiments of the subject matter previously described, the system and the various components and accessories that can be included in the system, e.g., reactants expander, can be made out of a plurality of suitable materials. Suitable materials include, but are not limited to, aluminum, stainless steel, carbon steel, glass-lined materials, polymer-based materials, nickel-based metal alloys, titanium-based alloys, cobalt-based metal alloys or combinations thereof. Additional non-limiting examples of suitable materials include chromium, hafnium, niobium, platinum, rare earth metals, rhenium, ruthenium, tantalum, titanium, tungsten and vanadium. In certain embodiments, such metals can be present within a metal alloy, e.g., a nickel-based alloy.

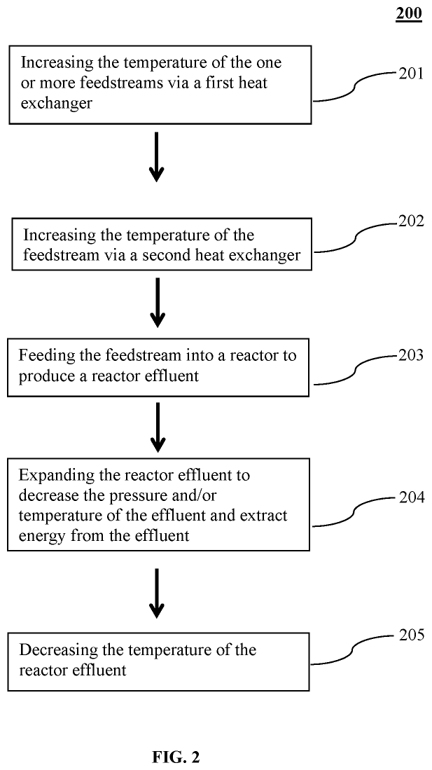

The presently disclosed subject matter provides methods for the production of olefins from a hydrocarbon feedstream and the extraction of heat energy from the hydrocarbon feedstream to drive downstream steps of the methods. For the purpose of illustration and not limitation, FIG. 2 is a schematic representation of a method according to a non-limiting embodiment of the disclosed subject matter. In certain embodiments, the method 200 can include providing one or more feedstreams. For example, the method 200 can include providing at least two feedstreams, e.g., a first feedstream and a second feedstream. In certain embodiments, the first feedstream can be a hydrocarbon stream, e.g., a liquid hydrocarbon stream. Non-limiting examples of such hydrocarbon streams include gas oil stream, naphtha streams, C4 hydrocarbon streams, ethane-containing streams and propane-containing streams. In certain embodiments, the second feedstream can be a gaseous feedstream, e.g., that includes hydrogen, steam, nitrogen or a combination thereof. In certain embodiments, the one or more feedstreams can include methane, gas oil, vacuum gas oil, vacuum residue, synthesis gas, Fischer-Tropsch liquids/waxes and/or pyrolysis gasoline. In certain embodiments, the pyrolysis gasoline can be obtained from a steam cracking process.

The method 200 can include increasing the temperature of the one or more feedstreams 201. In certain embodiments, the temperature of the one or more feedstreams can be increased by exchanging heat between the feedstreams and an additional stream to increase the temperature of the feedstreams. Heat exchange between the feedstreams and the additional stream can occur with a first heat exchanger. For example, the temperature of the feedstreams can be increased by the exchanging of heat with an additional stream such as, but not limited to, a flue gas or an effluent stream exiting a chemical reactor. In certain embodiments, prior to exchanging heat with the additional stream, the feedstreams can have a temperature from about 10° C. to about 150° C. In certain embodiments, the temperature of the feedstreams prior to heat exchange can be about 80° C. In certain embodiments, after exchanging heat with the additional stream, the temperature of the feedstreams can be of a temperature close to the boiling point of one or more components within the feedstream and/or of a temperature that results in the partial evaporation of the feedstream. In certain embodiments, the temperature of the feedstreams can be from about 300° C. to about 600° C. after heat exchange.

In certain embodiments where two or more feedstreams are subjected to heat exchange, the method 200 can further include combining the two or more feedstreams into a single feedstream (also referred to herein as a combined feedstream) and/or subjecting the single feedstream to a second heat exchange with the additional stream within the first heat exchanger (see FIG. 1). In certain embodiments, after exchanging heat with the additional stream for a second time, the temperature of the combined feedstream can be of a temperature that results in the evaporation of the feedstream, e.g., the heated combined feedstream can have a vapor fraction greater than about 90%, greater than about 91%, greater than about 92%, greater than about 93%, greater than about 94%, greater than about 95% or greater than about 96%. In certain embodiments, the one or more feedstreams can have a pressure of about 10 to about 50 bar absolute (bara) prior to and/or after heat exchange within the first heat exchanger. For example, and not by way of limitation, the one or more feedstreams can have a pressure of about 30 bara.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5% and/or up to 1% of a given value.

The method 200 can include further increasing the temperature of the feedstream 202. In certain embodiments, the temperature of the feedstream can be increased by heat exchange within a second heat exchanger (see FIG. 1). For example, and not by way of limitation, the heat exchanger can be located within a process furnace, e.g., within the convection section of the process furnace, and/or the feedstream can exchange heat with one or more gas streams within the furnace. In certain embodiments, the feedstream can exchange heat with one or more flue gases produced within the reactor, e.g., within the radiation section of the furnace (see FIG. 1). In certain embodiments, the feedstream can exchange heat with the reactor effluent and/or the feedstream can exchange heat with the exhaust gases (or the waste heat recovered therefore) of a turbine, e.g., a gas turbine. Following heat exchange within the second heat exchanger, the feedstream can have a temperature from about 500° C. to about 700° C.

Alternatively or additionally, prior to increasing the temperature of the feedstream within the second heat exchanger, the method can further include expanding the feedstream, e.g., the combined feedstream, to decrease the pressure and/or temperature of the feedstream. The feedstream can be expanded within an expander, e.g., an expander turbine as disclosed above. Following expansion, the feedstream can have a pressure from about 1 bara to about 20 bara, e.g., a pressure of about 4 bara, and can have a vapor fraction greater than about 90%, greater than about 91%, greater than about 92%, greater than about 93%, greater than about 94%, greater than about 95% or greater than about 96%. In certain embodiments, the temperature of the feedstream can be from about 300° C. to about 500° C. following expansion. In certain embodiments, methods of the disclosed subject do not include expanding the feedstream.

In certain embodiments, the method 200 can further include feeding the feedstream into a reactor to produce a product stream (also referred to herein as the reactor effluent) that includes the hydrocarbon reaction products 203. For example, and not by way of limitation, the methods of the disclosed subject matter can be used to produce a reactor effluent that include ethylene by using a hydrocarbon feedstream that include ethane and cracking the ethane within the reactor. In certain embodiments, the hydrocarbon feedstream can include butane and/or isobutane and/or the reactor effluent can include 1-butene, isobutylene, hydrogen or a combination thereof. In a non-limiting embodiment, the hydrocarbon feedstream can be a C4 hydrocarbon stream and/or the reactor effluent can include 1,3-butadiene. In certain embodiments, the hydrocarbon feedstream can be a kerosene fraction and/or the reactor effluent can include 1-butene, ethylene, methane, acetylene, ethane, propylene, propane, 1,3-butadiene, 1-butene, hydrogen or a combination thereof. In certain embodiments, the hydrocarbon feedstream can include propane and/or the reactor effluent can include hydrogen, methane, ethane, ethylene, propane, propylene or a combination thereof. In certain embodiments, the feedstream can include methane and steam and/or the reactor effluent can include hydrogen, carbon monoxide, carbon dioxide and/or steam. In certain embodiments, the feedstream can include pyrolysis gas and/or hydrogen and/or the reactor effluent can include hydrogen, methane, ethane, propane, butane, pentane, benzene, toluene, xylene and/or ethyl benzene. In certain embodiments, the feedstream can include naphtha, gas oil, vacuum gas oil and/or vacuum residue and/or the reactor effluent can include hydrogen, methane, ethane, ethylene, propylene, 1-butene, 2-butene, 1,3-butadiene, pyrolysis gas (pygas) and/or C9+ hydrocarbons.

In certain embodiments, the reactor and/or the combustion air can be heated using the radiant heat produced by the process furnace, e.g., produced within the radiation section of the furnace. For example, and not by way of limitation, the reactor or components of the reactor can be positioned within the radiant section of the furnace. In certain embodiments, the reactor effluent exiting the reactor can have a temperature of about 750° C. to about 880° C. and/or a pressure of about 2 bara to about 5 bara. In certain embodiments, the reactor effluent can have a temperature of about 850° C. and/or a pressure of about 3 bara. In certain embodiments, the reactor effluent can have a temperature of about 850° C. and/or a pressure in the range of about 2 bara to about 45 bara.

In certain embodiments, the method 200 can include expanding the reactor effluent in a reactor effluent expander to decrease the temperature and/or pressure of the reactor effluent 204 (see FIG. 1). In certain embodiments, upon expansion, the temperature of the reactor effluent can be decreased to about 600° C. to about 700° C., e.g., 630° C., and/or have a pressure from about 1.2 bara to 0.2 bara, e.g., 0.3 bara. The temperature and/or pressure of the reactor effluent exiting the reactor effluent expander can depend on the amount of cooling required, the inlet pressure of the reactor effluent and/or the inlet temperature of the reactor effluent. In the disclosed subject matter, the energy obtained during expansion of the reactor effluent in the expander can be converted into mechanical energy that can be used to drive other system components used within the disclosed methods. For example, and not by way of limitation, the extracted energy can be used to drive a compressor, e.g., in common drive with the reactants expander and/or the reactor effluent expander, which can be used to compress the reactor effluents that are produced within a chemical reactor used in the disclosed method. Non-limiting examples of methods for transferring the work obtained from the expansion of the feedstream and/or reactor effluents, described below, can include the use of gears, an electric generator, an electric motor, a hydraulic pump and/or motor or a pneumatic pump and/or motor. In certain embodiments, the work can be transferred by coupling the system component to the same axis as the reactants expander (e.g., mechanical coupling).

In certain embodiments, the method 200 can include decreasing the temperature of the reactor effluent 205. In certain embodiments, the temperature of the reactor effluent can be decreased by exchanging heat within one or more heat exchangers. Following heat exchange, the reactor effluent can have a final temperature of about 20° C. to about 50° C. and/or a final pressure of about 0.1 bara to about 2 bara. In certain embodiments, the reactor effluent can undergo multiple heat exchanges to have a final temperature and/or pressure as described above. For example, and not by way of limitation, the reactor effluent can exchange heat with the one or more feedstreams within the first heat exchanger. Following heat exchange within the first heat exchanger, the reactor effluent can have a temperature from about 300° C. to about 100° C. and/or a pressure of about 3 bara to about 0.1 bara, e.g., a temperature of about 130° C. and/or a pressure of about 0.3. In certain embodiments, the reactor effluent exiting the first heat exchanger can be further cooled within a third heat exchanger (see FIG. 1), e.g., by direct or indirect water cooling. In certain embodiments, the reactor effluent can have a temperature of about 20° C. to about 30° C. and/or a final pressure of about 0.1 bara to about 1 bara upon exiting the third heat exchanger, e.g., a temperature of about 30° C. and/or a pressure of about 0.3 bara.

In certain embodiments, the reactor effluent can be subjected to heat exchange prior to expansion within the reactor effluent expander. For example, and not by way of limitation, the temperature of the reactor effluent can be reduced upstream from the reactor effluent expander. In certain embodiments, the temperature of the reactor effluent can be reduced by means of direct quenching with a gas or liquid or indirect quenching through heat exchange, e.g., by steam generation in a primary transfer line exchanger. Cooling the reactor effluent prior to transferring the reactor effluent to the reactor effluent expander can allow the expander to operate at a lower inlet temperature and under less severe operating conditions which can allow the reactor effluent expander to be constructed of different types of materials.

In certain embodiments, following the reduction in the temperature of the reactor effluent, the method can include separating the components within the reactor effluent that condensed, if any, upon cooling. For example, and not by way of limitation, the method can further include compressing the reactor effluent to increase the pressure of the effluent following the reduction in the temperature of the reactor effluent. In certain embodiments, the pressure of the reactor effluent following compression can be from about 0.3 bara to about 40 bara to allow downstream separation of the hydrocarbons products from the reactor effluent. The type of separation processes used in the disclosed method depends on the types of hydrocarbons that are present within the reactor effluent. Non-limiting examples of separation processes that can be used in the disclosed methods are disclosed in U.S. Pat. Nos. 5,979,177, 6,637,237 and 6,705,113, EP2326899 and WO 2010/016815. One non-limiting embodiment of a separation process that can be used in the disclosed methods for the production of ethylene includes washing the reactor effluent after compression, drying and cooling the reactor effluent in a cold box, e.g., cooling can be provided from cold recovery of the products after separation and the use of an methane, ethylene and propylene refrigerant system, to a temperature in the range of about −150° C. to about −180° C. The condensed reactor effluent stream can be separated into C1, C2, C3, C4 and C5+ fractions in a series of distillation columns that include a demethanizer, a deethanizer, a depropanizer and/or a debutanizer. The deethanizer can produce a C2 fraction containing acetylene, ethylene and ethane. In certain embodiments, the acetylene can be extracted or hydrogenated to ethylene and the ethylene and ethane can be separated from the C2 fraction in a C2 splitter distillation column.

The following example is merely illustrative of the presently disclosed subject matter and should not be considered as a limitation in any way.

### Example 1: Hydropyrolysis of a Kerosene Fraction to Produce Ethylene

This example provides details regarding a hydropyrolysis method for cracking a kerosene fraction using the disclosed subject matter as compared to a steam cracking process known in the art. A simulation using the software Aspen Plus version 8.2 (Aspen Technology, Inc.) was performed to model the method and the yields were obtained using the software COILSIM1D. As shown in FIG. 1, a liquid hydrocarbon feed (1) with a pressure of 30 bar absolute (bara) and an original temperature of 80° C. was heated close to the boiling point or partly evaporated, but for less than 70%, with heat originating from the reactor effluent by means of a heat exchanger (3). The liquid hydrocarbon stream contained straight run kerosene that has a boiling point range from 150° C. to 300° C. Following heat exchange within the heat exchanger (3), the hydrocarbon feedstream had a temperature of 300° C. A stream of hydrogen (2) that had a pressure of 30 bara and an original temperature of 80° C. was heated with heat originating from the reactor effluent by means of a heat exchanger system (3) to such a temperature that after mixing with the liquid or partly evaporated hydrocarbon feed in a mixing device (4), the mixture was fully evaporated. Following heat exchange within the heat exchanger (3), the hydrogen stream had a temperature of 300° C. The formed hydrogen-hydrocarbon mixture was further heated in a heat exchanger system (3) to a temperature high enough, e.g., 500° C., so that after expansion in a reactor feed expander (5) the outlet stream is 95% vapor or more.

The reactants exiting the reactor feed expander was reheated in the convection section of a process furnace by drawing heat from the hot flue gasses leaving the radiation section (7) of the furnace. After reheating the reactor feed to a temperature of 650° C., the hydrogen-hydrocarbon mixture was cracked in a tubular reactor heated by radiant heat in the radiant section (7) of a process furnace. The reactor effluent exiting this tubular reactor was at a temperature of 840° C. and at a pressure of 3.1 bara.

The reactor effluent was expanded in a reactor effluent expander (8) to a pressure in the range of 0.35 bara. The pressure can be higher if less cooling is required or if the inlet pressure was higher. It can also be lower if more cooling is required or more work needs to be generated. The remainder of the heat present in the reactor effluent that exit the reactor effluent expander (8) was recovered by the heat exchanger (3) to produce a reactor effluent with a temperature 130° C., which was further cooled to a temperature 30° C. by means of direct or indirect water cooling (9). Condensed components were separated out (10) and the gas was compressed in a compressor (11) to a higher pressure to allow for downstream separation. The compressor (11) included several stages with interstage coolers (12).

The reactor feed expander (5), reactor effluent expander (8) and all or some stages of the compressor (11) were mounted on the same axis to allow for the transfer of mechanical work between these devices.

Methods for steam cracking of a kerosene fraction known in the art include cracking 10 t/h of a kerosene fraction at 818° C. COT and 1.7 bara COP, with a dilution of 3.5 t/h of steam. In the disclosed hydropyrolysis method as shown in FIG. 2, 10 t/h of a kerosene fraction was cracked at 818° C. COT and 2.8 bara COP mixed with 1.0 t/h of hydrogen feed. In the state of the art process, a separate drive for the downstream compressor is required. The downstream compressor usually is a steam turbine with associated equipment, such as a condenser, a means of steam generation, etc. All this equipment is not required in the disclosed methods because the work provided by the reactor feed expander and reactor effluent expander is sufficient to compress the charge gas to a pressure in excess of 30 bara.

Furthermore, the presently disclosed method consumes less energy compared to methods known in the art. For example, in the disclosed method, 0.754 t/h of fuel was used (which is equivalent to 38 GJ/h) as compared to 41 GJ/h that was used in a method known in the art. Further, the energy requirements for the furnace plus charge gas compressor section was approximately 41/4.54=9.0 GJ per tonne ethylene plus propylene for the steam cracking process (method known in the art), whereas it is 38/4.91=7.7 GJ per tonne ethylene plus propylene for the disclosed hydropyrolysis process. Therefore, the methods of the disclosed subject matter were more energy efficient than methods known in the art.

In addition, use of hydrogen as the diluent instead of steam resulted in the production of light hydrocarbon components from the feed (Table 1). Although ethane was produced in both the steam cracking process as well as the hydropyrolysis process, it was assumed to be recycled in an ethane steam cracking furnace under suitable conditions for ethane steam cracking with 75 wt % ultimate yield of ethylene from ethane and is indicated as 0 wt % in Table 1. In addition, considering the disclosed hydropyrolysis method consumes hydrogen, the weight percent for hydrogen is indicated as negative (Table 1). As shown in Table 1, the yield of ethylene and propylene from a liquid feedstock using the disclosed method was higher compared to steam cracking. In addition, the use of hydrogen in the disclosed hydropyrolysis process as the diluent can suppress coke formation in the reactor tube.

TABLE 1

| Method | Steam cracking method | Hydropyrolysis method |
|---|---|---|
| ultimate yield | % wt | % wt |
| Hydrogen | 2.2 | −1.2 |
| methane | 14.9 | 23.0 |
| ethylene | 31.3 | 40.5 |
| Ethane | 0 | 0 |
| propylene | 14.1 | 8.7 |
| C2= + C3= total | 45.4 | 49.1 |

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various patents and patent applications are cited herein, the contents of which are hereby incorporated by reference herein in their entireties.

1. A method for producing olefins using a hydrocarbon feedstream and a gas feedstream including hydrogen, comprising:
(a) increasing the temperature of the hydrocarbon feedstream and the gas feedstream via a heat exchanger;
(b) combining the hydrocarbon feedstream and the gas feedstream and reacting the feedstreams to produce a reactor effluent comprising one or more olefins;
(c) expanding the reactor effluent in a reactor effluent expander to decrease one or more of a reactor effluent pressure or reactor effluent temperature;
(d) transferring the reactor effluent to the heat exchanger to increase the temperature of the feedstreams and/or decrease the temperature of the reactor effluent; and
(e) compressing the reactor effluent by utilizing reactor effluent expansion.

2. The method of claim 1, wherein the hydrocarbon feedstream comprises ethane and/or the reactor effluent comprises ethylene.

3. The method of claim 1, wherein the hydrocarbon feedstream comprises propane and/or the reactor effluent comprises propylene.

4. The method of claim 1, further comprising expanding the combined feedstream in a reactants expander to decrease the pressure and/or the temperature of the combined feedstream prior to feeding the combined feedstream into the reactor.

5. The method of claim 1, further comprising separating the olefins from the reactor effluent to generate an olefin-containing stream.

6. The method of claim 1, wherein the gas feedstream further comprises a gas selected from the group consisting of steam, nitrogen, methane, hydrogen, carbon dioxide and a combination thereof.

7. The method of claim 1, wherein the gas feedstream and/or the hydrocarbon feedstream has a pressure of about 10 to 50 bar absolute and/or a temperature of about 10° C. to about 100° C. prior to increasing the temperature of the hydrocarbon feedstream and the hydrogen gas feedstream via a first heat exchanger.

8. A method for producing ethylene using a feedstream including ethane and hydrogen, comprising:
(a) increasing the temperature of the feedstream via a heat exchanger to a temperature equal to or greater than about 500° C.;
(b) feeding the feedstream into a reactor and increasing the temperature of the hydrocarbon feedstream to a temperature from about 500° C. to about 700° C.;
(c) cracking the feedstream at a temperature from about 650° C. to about 880° C. to produce a reactor effluent comprising ethylene at a temperature from about 780° C. to about 880° C. and/or a pressure from about 2 to about 5 bar absolute;
(d) expanding the reactor effluent in an reactor effluent expander to decrease one or more of a pressure of the reactor effluent to about 0.2 to about 1.2 bar absolute or reactor effluent temperature to about 600° C. to about 700° C.;
(e) transferring the reactor effluent to the heat exchanger to increase the temperature of the feedstream and/or decrease the temperature of the reactor effluent; and
(f) compressing the reactor effluent by utilizing reactor effluent expansion.

9. The method of claim 8, further comprising expanding the feedstream in a reactants expander to decrease the pressure and/or the temperature of the feedstream prior to feeding the feedstream into the reactor.

10. The method of claim 8, further comprising separating ethylene from the compressed reactor effluent to produce an ethylene product stream.

11. The method of claim 8, wherein the temperature of the reactor effluent is about 20° C. to about 50° C. prior to compressing the reactor effluent.

12. A method for producing ethylene using a feedstream including ethane and hydrogen, comprising:
(a) increasing the temperature of the feedstream via a heat exchanger;
(b) feeding the feedstream into a reactor and increasing the temperature of the feedstream;
(c) cracking the feedstream at a temperature from about 650° C. to about 880° C. to produce a reactor effluent comprising ethylene;
(d) expanding the reactor effluent in an reactor effluent expander to decrease one or more of a reactor effluent pressure or reactor effluent temperature;
(e) transferring the reactor effluent to the heat exchanger to increase the temperature of the feedstream and/or decrease the temperature of the reactor effluent;
(f) separating one or more condensed components from the reactor effluent; and
(g) compressing the reactor effluent by utilizing reactor effluent expansion.

13. The method of claim 12, further comprising separating ethylene from the compressed reactor effluent to produce an ethylene product stream.

14. The method of claim 12, further comprising decreasing the pressure and/or temperature of the feedstream in a reactants expander before feeding the feedstream into a reactor.

15. A system for producing olefins using a hydrocarbon feedstream and a gas feedstream including hydrogen, comprising:
(a) a first heat exchanger for increasing the temperature of the hydrocarbon feedstream and the hydrogen gas feedstream;
(b) a second heat exchanger, coupled to the first heat exchanger, for increasing the temperature of the feedstreams;
(c) a reactor, coupled to the second heat exchanger, for producing a reactor effluent from the feedstreams;
(d) a reactor effluent expander, coupled to the reactor and the first heat exchanger, for decreasing the temperature and/or pressure of the reactor effluent;
(e) a third heat exchanger, coupled to the first heat exchanger, for decreasing the temperature of the reactor effluent; and
(f) a compressor, coupled to the third heat exchanger and the reactor effluent expander, for compressing the reactor effluent in a compressor,
wherein the expansion of the reactor effluent drives the compressor.

16. The system of claim 15, further comprising a reactants expander, coupled to the first heat exchanger and the second heat exchanger, for decreasing the pressure and/or temperature of the feedstreams before entering the second heat exchanger.

17. The system of claim 15, wherein the system does not comprise a reactants expander.

18. A system for producing olefins using a hydrocarbon feedstream and a gas feedstream including hydrogen, comprising:
(a) a first heat exchanger for increasing the temperature of the hydrocarbon feedstream and the hydrogen gas feedstream;
(b) a reactants expander, coupled to the first heat exchanger, for decreasing the pressure and/or temperature of the feedstreams;
(c) a second heat exchanger, coupled to the reactants expander, for increasing the temperature of the feedstreams via a second heat exchanger;
(d) a reactor, coupled to the second heat exchanger, for producing a reactor effluent from the feedstream;
(e) a reactor effluent expander, coupled to the reactor and the first heat exchanger, for decreasing the temperature and/or pressure of the reactor effluent;
(f) a third heat exchanger, coupled to the first heat exchanger and the reactor effluent expander, for decreasing the temperature of the reactor effluent; and
(g) a compressor, coupled to the third heat exchanger, the reactor effluent expander and the reactants expander, for compressing the reactor effluent in a compressor,
wherein the expansion of the feedstreams and/or the reactor effluent drives the compressor.

19. The system of claim 18, wherein the reactants expander is coupled to the compressor and expansion of the feedstreams drives the compressor.

20. The system of claim 18, wherein the reactants expander, the reactor effluent expander and the compressor are coupled and the expansion of the feedstreams and the reactor effluent drives the compressor
